(19) United States
(12) Patent Application Publication
(10) Pub. No.: US 2026/0306666 A1
(43) Pub. Date: Oct. 8, 2026

(54) ADENOVIRUS VECTOR CIRCULAR RNA EXPRESSION SYSTEM

(71) Applicant: CIRCIO AB, Hässelby (SE)

(72) Inventors: Eoghan O'LEARY, Hässelby (SE); Thomas Birkballe HANSEN, Hässelby (SE)

(21) Appl. No.: 19/474,955

(22) PCT Filed: Apr. 12, 2024
(86) PCT No.: PCT/EP2024/060065

§ 371 Date: Oct. 13, 2025

(30) Foreign Application Priority Data
Apr. 14, 2023 (EP) 23167999.4

(51) Int. Cl. A61K 48/00; C12N 15/86

(52) U.S. Cl. CPC A61K 48/0041; C12N 15/86; C12N 2710/10321; C12N 2710/10343; C12N 2840/203

(57) ABSTRACT

The present invention relates to an adenovirus genome comprising a heterologous expression cassette encoding a circular RNA and its uses.

The present invention relates to an adenovirus genome comprising a heterologous expression cassette encoding a circular RNA.

### BACKGROUND OF THE INVENTION

Adenoviruses (AdV) are non-enveloped double-stranded DNA vectors. AdVs are capable of infecting a wide range of cell types and unlike retroviruses, these vectors are non-integrating. Thus, AdVs are an attractive vehicle for gene transfer with applications in vaccination, cancer treatment and gene therapy. Adenoviruses have many characteristics that make them attractive vectors for transgene delivery: adenovirus biology has been extensively studied; adenovirus genomes are relatively easy to manipulate and they are capable of carrying relatively large amounts of exogenous DNA. They have the capacity to infect both dividing and non-dividing cells, facilitating their use for a variety of purposes. Additionally, AdVs can induce humoral and cellular immune responses, which is beneficial for some aspects in the development of vaccines and in immune-oncology treatments.

Most AdVs are genetically modified versions of human Mastadenovirus, in particular the adenovirus serotype 5 genome. Different versions of AdVs exist, but they can be generally divided into 2 categories based on their capacity to replicate; conditionally replicative (CR) and replication deficient (RD). Conditionally replicating adenovirus (crAdV) have been employed for example for cancer treatment to specifically target and replicate in cancer cells, also known as oncolytic adenoviruses. The lytic nature of adenovirus replication directly kills infected cells, releasing the associated antigens which are recognised by the host immune system. Additionally, upon lysis progeny viruses can then be spread throughout the affected tissue, such as a tumor, further infecting, and destroying other affected cells.

The construction of crAdVs is based on the deletion or modification of viral gene functions that are critical to viral replication in normal cells, but are dispensable in the targeted cells. This includes insertion of mutations in the E1A region, or deletion of E1B from the wild-type genome, to target cancer cells with defects in the retinoblastoma (Rb) and p53 pathways, respectively, as these pathways are defective in most human tumors. Another strategy for targeting crAdVs replication in cancer cells involves the control of E1 transcription by introducing tumor- or tissue-specific promoters such as prostate-specific enhancer/promoter for prostate cancer, or E2F-I for cancer cells with a defective Rb pathway. Additionally, many crAdVs contain partial or full E3 region deletions. The E3 region encodes several genes involved in preventing infected cells from elimination by the immune system. Deletion experiments have determined that this region has been not essential for Ad replication in cell culture or in vivo. Thus, removal of these genes does not interfere with the replication ability of the adenovirus and increases the transgene insertion capacity of the adenoviral vector.

Several regions within the adenovirus genome have been identified where a desired transgene can be inserted. Dependent on the insertion site the transgene can be transcribed under the control of adenoviral promoters. Additionally, transgene expression cassettes can be incorporated within the adenovirus vector under the control of exogenous promoters, such as the high activity CMV promoter or tissue-specific promoters. Frequently, in E3 partial/full-region deletion AdVs, transgene expression cassettes are incorporated into the position of the E3 deleted gene/region. Many studies have shown that insertion of the transgene cassette under the control of the adenoviral E3 promoter or an exogenous promoter gives rise to high yield transgene expression and functional viruses.

While AdVs have been shown as powerful vehicles for transgene delivery, one limitation of the use of AdVs for transgene delivery is that the expression of the transgene is coupled to the presence of the virus. Due to the presence of pre-existing neutralising antibodies and induction of strong anti-viral innate immune responses upon treatment with AdVs, these vectors can be cleared relatively quickly and efficiently from the host, thus limiting the duration at which the transgene is expressed. Thus, to improve duration of transgene expression from AdVs without further interfering with the adenoviral genome the present invention provides AdVs capable of encoding circular RNA transcripts to deliver transgene payloads as opposed to conventional linear mRNA transcripts.

Circular RNA (circRNA) constitutes a novel class of RNAs just recently recognized. CircRNAs are characterized as covalently closed molecules typically produced by a non-linear ‘backsplicing’ event using a downstream splice donor (SD) and an upstream splice acceptor (SA) in contrast to conventional linear splicing. Endogenous circular RNA has been studied intensively in the past decade, and while some controversies on functional relevance exists, it is widely appreciated that circRNAs by virtue of their circular nature are resistant towards exo-nucleolytic decay, and therefore circRNAs comprise a very stable class of RNA with half-lives greatly exceeding that of conventional linear mRNA. Moreover, knowledge of circRNA production, both in vitro and in vivo, has improved significantly, and consequently, the therapeutic potential of a durable circular RNA concept with engineered functionalities has just recently emerged. In broad terms, circRNA production can be achieved by two distinct mechanisms, either 1) using group-I-intron derived ribozymatic circularization shown effective for in vitro production, or 2) using spliceosome-based backsplicing similar to endogenous circRNA biogenesis useful for in vivo production. In the latter setup, inserting flanking inverted element is known to stimulate backsplicing dramatically by positioning the splice sites involved in close proximity. While circRNAs are devoid of 5′cap and 3′ polyA tails and thus per se not substrates for translation, the insertion of internal ribosome entry sites (IRES) effectively converts non-coding circRNAs into a highly effective and durable protein-encoding molecules.

Due to circular RNAs intrinsic stability and their capacity to be modified to express protein, circular RNAs are an attractive payload to be introduced into adenoviruses. Engineering circRNA to express therapeutic proteins and expressing them from AdVs has the potential to further enhance adenovirus associated anti-tumour responses and improve their therapeutic potential in cancer treatment, but also other therapeutic applications. Moreover, several immunotherapeutic agents have shown to be toxic upon systemic delivery. In these cases, using a conditional oncolytic adenovirus that specifically replicates in the tumour-microenvironment restricts the expression of therapeutic protein in questions with high efficacy and improved safety.

However, surprisingly expression cassettes encoding circular RNA cannot be inserted in the same way as expression cassettes encoding, for example, linear mRNAs. The structural peculiarities of circular RNA encoding sequences as well as the other elements such as those required for circRNA formation and translation, requires the insertion at specific regions of the adenovirus to yield viable virus. The present invention teaches integration sites usable for circular RNA encoding constructs. Furthermore, the inventors surprisingly identified integration sites resulting in particularly high expression levels of circRNA.

### SUMMARY OF THE INVENTION

In a first aspect, the present invention provides an adenovirus genome encoding an adenovirus, comprising at least one heterologous expression cassette comprising or consisting of a nucleic acid sequence encoding a circular RNA (circRNA), wherein the heterologous expression cassette is inserted outside of functional transcriptional units of the adenovirus genome having the same reading direction as the inserted heterologous expression cassette.

### LIST OF FIGURES

In the following, the content of the figures comprised in this specification is described. In this context please also refer to the detailed description of the invention above and/or below.

FIG. 1: Insertion in region of deleted genes refers to the experiment of example 1. A) depicts the elements of the circRNA cassette, promoter, inverted repeat (IR), splice acceptor (SA), the open reading frame (ORF) used is a split-ORF that flanks the CVB3 IRES element. B) depicts the mRNA expression cassette used as a control. It differs from the circRNA cassette by the lack of inverted repeat elements and the IRES sequence. The mRNA ORF is arranged as a continuous ORF, while the circRNA is arranged in a split-ORF design. C) depicts a diagram of the Ad5 genome used indicating the main transcriptional units of the Ad5 genome and their reading direction. The elements at both ends of the genome are the ITRs. V1 indicates the insertion site and orientation of the inserted circRNA or mRNA expression cassette.

FIG. 2: Insertion between transcriptional units refers to the experiments disclosed in Example 2. A) depicts a schematic of the Ad5 genome indicating the transcriptional units E1A, E1B, E2B, L1-5, E3A/B and E4. Furthermore the ITR is indicated at each end of the adenovirus genome. Below and indicated by the drawn boxes the insertion sites for the expression cassettes are indicated (V1 to V9). The arrows for the transcriptional units and the expression cassettes indicate orientation of the elements. B) and C) depict immunoblot analysis of the tested constructs that resulted in viable virus (i.e. V2, V3, V4, V5, V8 and V9) showing detection of the encoded circRNA 24 hours (B)) and 48 hours (C)) post infection of the host cells (A549 cells) using equal number of virus particles.

FIG. 3: Schematic of adenovirus 5 genome. The main transcriptional units and their orientation is indicated by broad arrows. Gene names appear above each transcriptional unit. In the adenovirus genome used in the examples the gene names in italics for the E3 region (i.e. 6.7k, gp19k, ADP, RIDα/β and 14.7k) have been deleted. Smaller arrows below the schematic (i.e. V1 to V9) represent insertion points and orientation of heterologous expression cassettes. Filled arrows (i.e. V2, V3, V4, V5, V8 and V9) depicts insertion point where viable virus was recovered and unfilled arrows (i.e. V1, V6 and V7) depicts insertion point where no viable virus was recovered.

FIG. 4: Alignment of human adenovirus. Depicted is an alignment of the Mastadenovirus family genomes showing the indicated transcriptional units/genes. The available adenovirus genomes of the family have been grouped by species and then only one genome per species is shown. The general genomic organization is highly conserved throughout the family. The relative position of the transcriptional units to each other is identical throughout the family. In consequence the insertion points identified in Ad5 (adenovirus genome used is depicted as ONCOS-d2.7 kb) are conserved in this family of adenovirus and insertion at the same relative positions would also result in insertion outside of transcriptional units. The insertion sites disclosed in the example section have been indicated in the ONCOS-d2.7 kb genome as black triangles ▴ (V5), plus operator + (V6/V7), unfilled rectangle □ (V1/V2), crossed-out box (V3/V4) or filled box ▪ (V8/V9).

### DETAILED DESCRIPTIONS OF THE INVENTION

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, Leuenberger, H. G. W, Nagel, B. and Klbl, H. eds. (1995), Helvetica Chimica Acta, CH-4010 Basel, Switzerland).

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. In the following passages, different aspects of the invention are defined in more detail. Each aspect so defined may be combined with any other aspect or aspects unless clearly indicated to the contrary. In particular, any feature indicated as being optional, preferred or advantageous may be combined with any other feature or features indicated as being optional, preferred or advantageous.

Several documents are cited throughout the text of this specification. Each of the documents cited herein (including all patents, patent applications, scientific publications, manufacturer's specifications, instructions etc.), whether supra or infra, is hereby incorporated by reference in its entirety. Nothing herein is to be construed as an admission that the invention is not entitled to antedate such disclosure by virtue of prior invention. Some of the documents cited herein are characterized as being “incorporated by reference”. In the event of a conflict between the definitions or teachings of such incorporated references and definitions or teachings recited in the present specification, the text of the present specification takes precedence.

In the following, the elements of the present invention will be described. These elements are listed with specific embodiments; however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

### Definitions

In the following, some definitions of terms frequently used in this specification are provided. These terms will, in each instance of its use, in the remainder of the specification have the respectively defined meaning and preferred meanings.

As used in this specification and the appended claims, the singular forms “a”, “an”, and “the” include plural referents, unless the content clearly dictates otherwise.

The term “about” when used in connection with a numerical value is meant to encompass numerical values within a range having a lower limit that is 5% smaller than the indicated numerical value and having an upper limit that is 5% larger than the indicated numerical value.

The term “adenovirus genome” as used herein refers to the genetic material of an adenovirus. Adenoviruses are double-stranded DNA viruses, and their genomes are relatively large compared to many other viruses, with around 30-40 kilobase pairs of DNA. The adenovirus genome contains several genes that are responsible for viral replication, assembly, and release. These genes encode for proteins that are essential for the virus to complete its life cycle, such as the polymerase that copies the viral DNA, the capsid proteins that form the protective shell around the virus, and the fiber proteins that help the virus attach to and enter host cells. An adenovirus genome also contains non-coding regions that regulate gene expression and replication, including promoters, enhancers, and origins of replication. These regulatory regions are important for controlling when and how adenovirus genes are expressed, and for ensuring that the virus replicates efficiently.

In the adenovirus genome, genes can be broadly categorized into early, intermediate and late genes based on their timing of expression during the viral replication cycle. Early genes (E) are expressed early during infection and are primarily involved in regulating viral replication and evading host immune responses. Intermediate genes are typically transcribed following transcription of early genes while late genes (L) are expressed late during infection and primarily encode the structural proteins that make up the viral capsid.

The adenovirus genome comprises the following genes that may be divided into further subgroups, early genes E1 to E4, intermediate genes pIX and pIVa2 and late genes L1 to L5. These genes may be further subdivided into E1A, E1B, E2A, E2B, E3A/B and E4. The late (L) genes are under the control of the MLP promoter sequence and are spliced to give rise to specific proteins. The E2 genes are considered to be regulated by multiple promoter/enhancer sequences. The different genes in the adenovirus genome may be coded on different strands of the double stranded genome or may even overlap if encoded on the same strand.

The term “conditionally replicating adenovirus” (crAdV) as used herein refers to a type of adenovirus that has been genetically modified to selectively replicate only under specific conditions, such as for example those presented in a particular cell type or tissue, such as cancer cells. CrAdVs are designed to have mutations or deletions in one or more genes that are essential for viral replication. However, these mutations or deletions are compensated for by the presence or absence of certain cellular factors or genetic alterations that are specific to the cells in which the crAdV can replicate, such as e.g. cancer cells. This allows the crAdV to selectively replicate and lyse in the specific cells (e.g. cancer cells) while sparing cells that do not provide these additional factors.

The term “mastadenovirus” as used herein refers to a genus of the Adenoviridae family, which infects a wide range of vertebrate hosts, including humans, other mammals, birds, and reptiles. Mastadenoviruses are further classified into seven species (A to G) based on their genetic and antigenic properties, as well as their host range and tissue tropism. Humans are known to be susceptible to several mastadenovirus species, including human mastadenovirus A to G, which can cause a variety of clinical manifestations, such as respiratory illness, gastroenteritis, conjunctivitis, and hemorrhagic cystitis. In a preferred embodiment of the present invention the adenovirus genome is a mastadenovirus genome. In a preferred embodiment of the present invention the adenovirus genome is a human mastadenovirus C, more preferably Adenovirus serotype 5 (Ad5).

The term “transcriptional unit” as used herein refers to a segment of DNA that contains all the genetic elements necessary for the transcription of one or more genes into RNA. The transcriptional unit in the context of the present invention therefore typically comprises or consists of a TATA box, one or more exons (encoding a gene), a polyadenylation signal and optionally one or more introns. In some instances the transcriptional units are delimited by other elements than a TATA box and instead rely on other elements that initiate transcription. Preferably the transcriptional unit comprises i) a TATA box or another promoter element, preferably having a sequence selected from CTTAAG or CCAAT, ii) one or more exons, iii) a polyadenylation signal and iv) optionally one or more introns.

In a preferred embodiment the transcriptional unit is delimited by a TATA box and a PAS sequence.

Non-limiting examples of transcriptional units of the adenovirus genome are the early genes E1 to E4, intermediate genes pIX and pIVa2 and late genes L1 to L5. Table 2 provides a list of transcription units present in human adenovirus 5 used in the examples. In a preferred embodiment the adenovirus genome comprises the transcription units E1A, E1B, pIX, pIVa2, E2A, E2B, L1-L5, E3 and E4.

The term “TATA box” as used herein refers to a DNA sequence that plays a crucial role in the initiation of transcription of protein-coding genes in eukaryotes. It is typically located upstream of the transcription start site, and it is recognized by the transcription factor IID (TFIID) complex, which is a component of the RNA polymerase II preinitiation complex. The TATA box is characterized by the presence of a conserved sequence of nucleotides. The consensus sequence of the TATA box comprises 5′-TAT (A/T) (A/T)-3′ or 5′-TAT (A/T) (A/T) A-3′. Preferably the sequence of the TATA box is selected from TATTTATA, TATATAA, TATATAAA, TATAA, TATATA.

The term “polyadenylation signal” is used interchangeably with the terms “poly(A) signal” or “PAS” and herein refers to a DNA sequence located downstream of the coding sequence in genes that signals the addition of a poly(A) tail to the 3′ end of the mRNA during transcription. The sequence of the polyadenylation signal is typically 5′-AATAAA-3′ or 5′-ATTAAA-3′. In a preferred embodiment the PAS has the sequence 5′-AATAAA-3′. For example the transcription units E1A, pIX, pIVa2, E3, L5 and E4 comprise the PAS having the sequence 5′-AATAAA-3′

The terms “5′” and “3′”, as used herein, identify one end of a single-stranded nucleic acid molecule. The 5′ end is that end of the molecule which terminates in a 5′ phosphate group and the 5′ direction is the direction toward the 5′ end. Likewise, the 3′ end is that end of the molecule which terminates in a 3′ hydroxyl group and the 3′ direction is the direction toward the 3′ end. In the context of the present invention nucleic acid sequences are written with the 5′ end to the left and the 3′ end to the right (unless noted otherwise), in reference to the direction of DNA synthesis during replication (from 5′ to 3′), RNA synthesis during transcription (from 5′ to 3′), and the reading of mRNA sequence (from 5′ to 3′) during translation.

The term “reading direction” as used herein refers to the direction of transcription. Transcription proceeds in a 5′ to 3′ direction meaning that RNA polymerase reads the DNA template strand from the 3′ end to the 5′ end and synthesizes the complementary RNA molecule in the 5′ to 3′ direction. The reading direction with regard to the adenovirus genome of the present invention thus refers to the 5′ to 3′ direction a particular gene is transcribed in. This applies to the DNA forward strand as well as to the reverse strand, wherein in both cases transcription proceeds in 5′ to 3′ direction in relation to the coding DNA strand.

The terms “upstream” and “downstream” as used herein refer to a relative location of one nucleic acid sequence with respect to another on a particular nucleic acid molecule.

Specifically, upstream refers to the nucleotide sequence that is located in the direction towards the 5′ end of the DNA strand. This means that upstream is located “upstream” of the gene or nucleotide sequence of interest, and is located in the opposite direction of transcription.

Likewise, downstream refers to the nucleotide sequence that is located in the direction towards the 3′ end of the DNA strand. This means that downstream is located “downstream” of the gene or nucleotide sequence of interest, and is located in the same direction of transcription.

The term “distance” as used herein refers to the number of nucleotides (in base pairs (bp)) that are between two nucleic acid sequences (or specific points such as an insertion point) located in the same nucleic acid molecule. For example, the two nucleic acid sequences X₁ and X₂ in the sequence X₁ATGCCAATGCX₂ have a distance of 10 base pairs, because 10 nucleotides are located between these sequences.

The term “inverted terminal repeat” (ITR) as used herein refers to a specific sequence of DNA found at both ends of the linear, double-stranded DNA genomes of many viruses, including adenoviruses. The ITR is a short sequence that is repeated in an inverted orientation, meaning that the sequence at one end is complementary to the sequence at the other end, and the two ends can anneal together to form a hairpin structure.

The term “heterologous”, as used herein (in particular in context of an expression cassette), refers to a genetic element that is derived from a different species or source than the one it is being compared or used with. For example a heterologous expression cassette would refer to an expression cassette that contains or consists of elements not derived from the same species that it is inserted to.

The term “open reading frame”, as used herein, refers to a portion of a nucleic acid sequence between the start and stop codons, which does not include a stop codon (which functions as a stop signal). A codon is a DNA or RNA sequence of three nucleotides that forms a unit of genomic information encoding a particular amino acid or signalling the termination of protein synthesis (stop codon). In the standard genetic code three different stop codons are known TAG, TAA or TGA on the DNA level or UAG, UAA and UGA on the RNA level. In variations of the standard genetic code other stop codons may exist. A start codon is the first codon of an RNA transcript (e.g. linear mRNA, circRNA) translated by a ribosome. Typically, the start codon has the sequence AUG. In rare instances the start codon may also have the sequence NUG (with N being any nucleotide).

The term “backsplicing”, as used herein, refers to splicing at a reversed order (i.e. backsplicing) in which an upstream 3′ splicing site is joined with a downstream 5′ splicing site.

The term “backsplicing site”, as used herein, refers to a nucleotide sequence at the intron exon boundary (i.e. a splice site). The sequence of the backsplicing site typically comprises two half-sites, whereas one half site is located on the intron and the other half-site on the exon. The spliced nucleotide sequence is split between the two half-sites of the backsplicing sites.

The term “internal ribosomal entry site (IRES)” refers to regions in the RNAs that allow the internal initiation of translation in a cap-independent manner. An IRES element typically comprises a stretch of highly structured RNA containing several stem-loop structures. IRESs have initially been identified in picornavirus, but they are present in a variety of different virus. More recently IRES sequences have also been identified in many cellular mRNAs. Both types of IRES sequences (i.e. viral and cellular IRES sequences) generally can be used to practice the present invention.

The term “pharmaceutically acceptable” means approved by a regulatory agency of the Federal or a state government or listed in the U.S. Pharmacopeia, European Pharmacopeia (Ph. Eur.) or other generally recognized pharmacopeia for use in animals, and more particularly in humans.

The term “carrier”, as used herein, refers to a diluent, adjuvant, excipient, or vehicle with which the therapeutic agent is administered. Such pharmaceutical carriers can be sterile liquids, such as saline solutions in water and oils, including those of petroleum, animal, vegetable or synthetic origin, such as peanut oil, soybean oil, mineral oil, sesame oil and the like. A saline solution is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsions, tablets, pills, capsules, powders, sustained-release formulations and the like. The composition can be formulated as a suppository, with traditional binders and carriers such as triglycerides. The compounds of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the patient. The formulation should suit the mode of administration.

### EMBODIMENTS

In the following different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

In a first aspect, the present invention provides an adenovirus genome encoding an adenovirus, comprising at least one (preferably heterologous) expression cassette comprising or consisting of a nucleic acid sequence encoding a circular RNA (circRNA), wherein the (preferably heterologous) expression cassette is inserted outside of functional transcriptional units of the adenovirus genome having the same reading direction as the inserted (preferably heterologous) expression cassette.

The inventors have surprisingly found that inserting expression cassettes encoding circRNA into the adenoviral genome may not allow the formation of adenovirus depending on the insertion point of the expression cassette. This is different to the insertion of expression cassettes that do not encode circRNA and instead encode elements such as mRNA. Without wishing to be bound by theory the inventors believe that this incompatibility of expression cassettes encoding circRNA may be related to the specific structure of the circRNA and related elements such as splice sites (i.e. splice acceptor and splice donor elements) and IRES elements that may prevent formation of viral particles. Specifically, the splicing sites of circRNA expression cassettes that are required for in vivo circRNA circularization in a backsplicing event, may interfere with the splice acceptor/donor sites of the adenovirus genome and thus result in unfavorable splicing events. Inserting an expression cassette outside of a functional transcriptional unit of the adenovirus as disclosed herein was found to result in functional adenovirus. The heterologous expression cassette is inserted outside of functional adenoviral transcriptional units that have the same reading direction as the inserted heterologous expression cassette. For example, a heterologous expression cassette inserted in orientation V1 as exemplified in the examples would be inserted within a transcriptional unit that has the same orientation (i.e.E3A/B). Whereas a heterologous expression cassette inserted in orientation V2 (i.e. at the same point as V1 but in the opposite direction) would be inserted outside of a functional adenoviral transcriptional unit that has the same reading direction.

In a preferred embodiment, the heterologous expression cassette is not inserted between the 5′-terminal ITR and E1A, between E2A and E3 and between pIX and pIVa2.

In a preferred embodiment, the heterologous expression cassette is not inserted between the 5′-terminal ITR and E1A.

In a preferred embodiment, the heterologous expression cassette is not inserted between E2A and E3.

In a preferred embodiment, the heterologous expression cassette is not inserted between pIX and pIVa2.

In a preferred embodiment, the expression cassette is a heterologous expression cassette, wherein the genetic elements such as the encoded circRNA and/or other elements of the expression cassette do not originate from the adenovirus genome used.

In a preferred embodiment, the transcriptional units referred to are the early genes E1 to E4, the intermediate genes pIX and pIVa2 and late genes L1 to L5.

In a preferred embodiment, the transcriptional units referred to are those indicated in Table 2 below.

In another preferred embodiment the transcriptional unit are the transcriptional units encoding specific adenoviral proteins. Non-limiting examples are pIX and pIVa2. Further examples can be found in Table 2.

Furthermore, the location of the insertion point within the adenovirus genome does have a significant impact on the expression level of the encoded circRNA.

In a preferred embodiment, the heterologous expression cassette is inserted outside of functional transcriptional units of the adenovirus genome encoded on the same strand of the adenovirus genome in which the heterologous expression cassette (in particular the circRNA encoded therein) is encoded.

In a preferred embodiment, the heterologous expression cassette is inserted at a point outside of functional transcriptional units of the adenovirus genome encoded on either strand of the adenovirus genome. In another preferred embodiment the heterologous expression cassette is inserted outside of functional transcriptional units encoding early genes E1 to E4 encoded on either strand of the adenovirus genome.

In a preferred embodiment, the heterologous expression cassette is inserted at a point between functional transcriptional units encoded on either strand of the adenovirus genome. Preferably the functional transcriptional units in between which the heterologous expression cassette is inserted are the closest functional transcriptional units, i.e. there are no other functional transcriptional units between them.

In a preferred embodiment of the first aspect of the present invention, the (preferably heterologous) expression cassette is not inserted between the transcriptional units pIX and pIVa2.

In a preferred embodiment of the first aspect of the present invention, the (preferably heterologous) expression cassette is inserted between a functional transcriptional unit and the ITR of the adenovirus genome, wherein no other functional transcriptional unit is between the functional transcriptional unit and the ITR.

In a preferred embodiment of the first aspect of the present invention, the (preferably heterologous) expression cassette is inserted.
a) between functional transcriptional units encoded on either strand of the adenovirus genome, wherein the functional transcriptional units in between which the heterologous expression cassette is inserted are the closest functional transcriptional units (i.e. there are no other functional transcriptional units between them); or
b) between a functional transcriptional unit and the ITR of the adenovirus genome, wherein no other functional transcriptional unit is between the functional transcriptional unit and the ITR.

In a preferred embodiment of the first aspect of the present invention, the adenovirus is a conditionally replicating adenovirus. In replication deficient adenovirus not all genes of the adenovirus genome are required to yield functional adenovirus. This provides more opportunities in selecting an insertion site within the adenovirus genome, because even if interference with some elements in the adenovirus genome occur as described above this is not relevant if elements are affected that are not essential in a replication deficient adenovirus. In consequence in a conditionally replicative adenovirus there are less suitable insertion sites available. The present invention however provides insertion sites taking these additional requirements into account and which are also functional in a conditionally replicating adenovirus.

In a preferred embodiment of the first aspect of the present invention, the heterologous expression cassette is inserted at a point in the adenovirus genome having a distance of at least 24 bp (at least 25 bp, at least 30 bp, at least 35 bp) to any transcriptional unit encoding an intermediate gene (preferably encoded on either strand of the adenovirus genome). Preferred examples of transcriptional units encoding an intermediate gene are pIX and pIVa2.

In a preferred embodiment of the first aspect of the present invention, the heterologous expression cassette has a distance of at least 24 bp upstream (i.e. 5′ direction) or at least 24 bp downstream (i.e. 3′ direction) to any functional transcriptional unit of the adenovirus genome.

In a preferred embodiment of the first aspect of the present invention, the distance of the heterologous expression cassette to the inverted terminal repeat (ITR) of the adenovirus genome is shorter upstream than downstream of the heterologous expression cassette.

The inventors have further observed that the orientation of the expression cassette with regard to the closest ITR of the adenovirus affects the expression strength of the circRNA encoded in the expression cassette. Expression cassettes having a reading direction towards the closest ITR have a lower expression level of the encoded circRNA as expression cassettes having a reading direction away from the closest ITR. Without wishing to be bound by theory, the inventors hypothesize that the regulatory elements located in the ITR may increase the transcription of the circRNA encoded in the expression cassette if in inserted in the correct orientation.

In a preferred embodiment of the first aspect of the present invention, the inverted terminal repeat (ITR) of the adenovirus genome is within 6000 nucleotides upstream of the heterologous expression cassette. In other words, the 5′ end of the heterologous expression cassette has a distance to the ITR in 5′ direction of 6000 nucleotides or less. In a preferred embodiment the distance to the ITR in 5′ direction is less than 5000, less than 4000, less than 3000 or less than 2000 nucleotides.

In a preferred embodiment of the first aspect of the present invention, the heterologous expression cassette is inserted between transcriptional units E1A/E1B, E4/E2A, L5/E4 or between E4 and the 3′-terminal ITR of the adenovirus genome (i.e. upstream of E4). The above transcriptional units may be encoded on different strands of the adenovirus genome.

In a preferred embodiment the end of transcriptional unit E1A is characterized by the PAS of E1A and the start of transcriptional unit E1B is characterized by the TATA box of E1B.

In a preferred embodiment the end of transcriptional unit E4 is characterized by the PAS of E4 and the start of transcriptional unit E2A is characterized by the promoter of E2A.

In a preferred embodiment the end of transcriptional unit L5 is characterized by the PAS of L5 and the end of transcriptional unit E4 is characterized by the PAS of E4.

In a preferred embodiment the start of transcriptional unit E4 is characterized by the TATA box of E4 and the 3′-terminal ITR of the adenovirus genome, which is located upstream of E4.

In a preferred embodiment, the (preferably heterologous) expression cassette is inserted between the transcriptional units that are delimited by the nucleic acid sequences as indicated in Table 1 below. Preferably the heterologous expression cassette is inserted between transcriptional units E1A/E1B, E4/E2A, L5/E4 or between E4 and the 3′-terminal ITR of the adenovirus genome (i.e. upstream of E4) as annotated in Table 1 below.

TABLE 1
Annotated TATA (or promoter) and PAS signals and
surrounding sequences SID refers to SEQ ID NO;
TATA box (or relevant promoter element) and PAS
sequence is underlined in above sequences.

| | Strand | Promoter | PAS |
|---|---|---|---|
| E1A | F | agctgacgtgtagtgTATTTAtacc | tgagttgatgtaagtttAATAAAgggtgagat |
| | | cggtgagttcct (SID 2) | aatgt (SID 10) |
| E1B | F | gggcttaaagggTATAtaatgcgcc | tgcggtttaaaacataAATAAAaaaccagact |
| | | gtgggct (SID 3) | ctgtt (SID 11) |
| pIX | F | gtgggaaagaataTATAaggtgggg | tgcggtttaaaacataAATAAAaaaccagact |
| | | gtcttatgta (SID 4) | ctgtt (SID 12) |
| pIVa2 | R | ctacacctacaaaCCAATcaccttc | gcaaaacccctaAATAAAgacagcaagacac |
| | | cttgatgcc (SID 5) | (SID 13) |
| L1-5 | F | (MLP) | (L5) |
| | | gaaggggggcTATAaaagggggtgg | gagaagatgactAATAAAgaatcgtttg |
| | | gggc (SID 6) | (SID 14) |
| E3 | F | cgcccgggcagggTATAactcacct | taaaaaaaaatAATAAAgcatcacttac |
| | | gac (SID 7) | (SID 15) |
| E2A/B | R | cgcgaaactagtcCTTAAgagtcag | gcaaaacccctaAATAAAgacagcaagacac |
| | | cgcg SID 8) | (SID 16) |
| E4 | R | tcattttttagtccTATAtatactc | tgcaattgaaaAATAAAcacgttgaaacat |
| | | gctctg (SID 9) | (SID 17) |

In a preferred embodiment of the first aspect of the present invention, the adenovirus genome is a Mastadenovirus genome.

In a preferred embodiment of the first aspect of the present invention, the adenovirus genome is a great ape Mastadenovirus genome.

In a preferred embodiment of the first aspect of the present invention, the adenovirus genome is selected from: African pygmy hedgehog, Alpaca, Bat, Bovine, Canine, Deer, Dolphin, Equine, Fowl, Frog, Guinea Pig, Lemur, Mandril, Murine, Ovine, Pigeon, Platyrrhini, Polar bear, Porcine, Reindeer, Rhesus, Sea lion, Simian, Skunk, Squirrel, Squirrel monkey, Tree shrew and Turkey, adenovirus genome.

In a preferred embodiment of the first aspect of the present invention, the adenovirus genome is a human Mastadenovirus genome.

In a preferred embodiment of the first aspect of the present invention, the adenovirus genome is a human Mastadenovirus group C genome.

In a preferred embodiment of the first aspect of the present invention, the adenovirus genome is a human adenovirus 5 (i.e. serotype 5; Ad5, HAdV-5) genome.

In a preferred embodiment of the first aspect of the present invention, the adenovirus genome is a human Mastadenovirus genome selected from HAdV-1, HAdV-105, HAdV-106, HAdV-11, HAdV-11+34, HAdV-11a, HAdV-12, HAdV-14, HAdV-16, HAdV-17, HAdV-18, HAdV-19, HAdV-2, HAdV-20, HAdV-21, HAdV-21a, HAdV-22, HAdV-23, HAdV-24, HAdV-25, HAdV-27, HAdV-29, HAdV-3+7, HAdV-30, HAdV-31, HAdV-32, HAdV-33, HAdV-35, HAdV-36, HAdV-38, HAdV-39, HAdV-40, HAdV-41, HAdV-42, HAdV-43, HAdV-44, HAdV-45, HAdV-47, HAdV-4a, HAdV-4p, HAdV-5, HAdV-51, HAdV-53, HAdV-54, HAdV-55, HAdV-56, HAdV-58, HAdV-6, HAdV-60a, HAdV-61, HAdV-62, HAdV-63, HAdV-64, HAdV-66, HAdV-68, HAdV-69, HAdV-7, HAdV-71, HAdV-7d, HAdV-7d2, HAdV-86, HAdV-89, HAdV-B3, HAdV-D10, HAdV-D13, HAdV-D37, HAdV-D8, HAdV-E4, HAdV-A, HAdV-B, HAdV-C, HAdV-D, HAdV-E, HAdV-F.

In a preferred embodiment of the first aspect of the present invention, the adenovirus genome is a human Mastadenovirus genome selected from African pygmy hedgehog adenovirus 1, Bat mastadenovirus, Bat mastadenovirus A, Bat mastadenovirus B, Bat mastadenovirus C, Bat mastadenovirus D, Bat mastadenovirus E, Bat mastadenovirus F, Bat mastadenovirus G, Bovine mastadenovirus B, Bovine mastadenovirus C, Canine mastadenovirus A, Deer mastadenovirus B, Dolphin mastadenovirus B, Eidolon helvum adenovirus, Equine mastadenovirus A, Equine mastadenovirus B, Guinea pig adenovirus, Human adenovirus 86, Human adenovirus sp., Human mastadenovirus A, Human mastadenovirus B, Human mastadenovirus C, Human mastadenovirus D, Human mastadenovirus E, Human mastadenovirus F, Human mastadenovirus G, Mastadenovirus sp., Murine mastadenovirus A, Murine mastadenovirus B, Murine mastadenovirus C, ONCOS-d2.7 kb, Ovine mastadenovirus A, Ovine mastadenovirus C, Platyrrhini mastadenovirus A, Polar bear mastadenovirus A, Porcine mastadenovirus A, Porcine mastadenovirus B, Porcine mastadenovirus C, Rhesus adenovirus 54, Rhesus adenovirus 55, Rhesus adenovirus 56, Rhesus adenovirus 57, Rhesus adenovirus 58, Rhesus adenovirus 59, Rhesus adenovirus 60, Rhesus adenovirus 61, Rhesus adenovirus 62, Rhesus adenovirus 63, Rhesus adenovirus 64, Rhesus adenovirus 65, Rhesus adenovirus 66, Rhesus adenovirus 67, Rousettus aegyptiacus adenovirus, Sea lion mastadenovirus A, Simian adenovirus B, Simian adenovirus ER, Simian mastadenovirus A, Simian mastadenovirus B, Simian mastadenovirus C, Simian mastadenovirus D, Simian mastadenovirus E, Simian mastadenovirus F, Simian mastadenovirus G, Simian mastadenovirus H, Simian mastadenovirus I, Skunk adenovirus HUN/2009, Skunk mastadenovirus A, Squirrel mastadenovirus A, Squirrel monkey adenovirus and Tree shrew mastadenovirus A.

In a preferred embodiment of the first aspect of the present invention, the (preferably heterologous) expression cassette comprises an open reading frame (ORF) encoding a circRNA of interest and an internal ribosome entry site (IRES) operably linked to the ORF.

In a preferred embodiment of the first aspect of the present invention, the (preferably heterologous) expression cassette comprises an open reading frame (ORF) encoding a circular RNA of interest and an internal ribosome entry site (IRES) operably linked to the ORF, a first backsplicing site that is positioned 5′ of the IRES, and a second backsplicing site that is positioned 3′ of the IRES.

In a preferred embodiment of the first aspect of the present invention, the (preferably heterologous) expression cassette further comprises a first inverted repeat (IR) element positioned 5′ of the first backsplicing site and a second IR element positioned 3′ of the second backsplicing site.

In a preferred embodiment of the first aspect of the present invention, the (preferably heterologous) expression cassette further comprises a promoter operably linked to the expression cassette to direct expression of the expression cassette.

In a preferred embodiment of the first aspect of the present invention, the (preferably heterologous) expression cassette further comprises a first inverted repeat (IR) element positioned 5′ of the first backsplicing site and a second IR element positioned 3′ of the second backsplicing site and a promoter operably linked to the expression cassette to direct expression of the expression cassette.

In a preferred embodiment of the first aspect of the present invention, the (preferably heterologous) expression cassette further comprises a polyadenylation signal.

In a preferred embodiment of the first aspect of the present invention, the ORF is a split ORF, wherein the IRES is flanked by the two parts of the split ORF.

In a preferred embodiment of the first aspect of the present invention, the IRES comprised in the (preferably heterologous) expression cassette the IRES is positioned within the (preferably heterologous) expression cassette in that:
(I) the 5′ end of the IRES and the 3′ end of the first backsplicing site are separated by at least 50 nucleotides, preferably by at least 200 more preferably by at least 300 nucleotides, and
(II) the 3′ end of the IRES and the 5′ end of the second backsplicing site are separated by at least 300 nucleotides, preferably by at least 350 nucleotides.

In a preferred embodiment of the first aspect of the present invention, the circular RNA of interest in the heterologous expression cassette is a transgene.

In a preferred embodiment of the first aspect of the present invention, the expression cassette has the sequence as indicated in SEQ ID NO: 001.

In a second aspect of the present invention an infectious adenovirus particle comprising the adenovirus genome of the first aspect of the invention is provided.

In a preferred embodiment of the second aspect of the present invention, the adenovirus particle is a conditionally replicative adenovirus.

In a preferred embodiment of the second aspect of the present invention, the adenovirus particle is a replication incompetent adenovirus.

In a third aspect of the invention a pharmaceutical composition comprising the adenovirus genome of the first aspect or the infectious adenovirus particle of the second aspect of the invention and further comprises a pharmaceutically acceptable carrier and/or excipient.

In a fourth aspect the present invention further provides for the medical use of the adenovirus genome according to the first aspect of the invention, the infectious adenovirus particle of the second aspect of the invention and the pharmaceutical composition according to the third aspect of the invention. The medical use encompasses the use in therapy and prophylaxis. Preferably for use in therapy and prophylaxis of proliferative diseases.

### EXAMPLES

The inventors observed that insertion of a circular RNA expression cassette within the adenoviral genome has significant effects on both transgene expression and adenoviral fitness. In the following examples the identification of insertion sites with an adenovirus genome suitable for the expression of a circRNA encoding expression cassette are provided. In particular sites resulting in superior expression strength of the encoded circRNA are provided.

### Example 1: Insertion at Site of Deleted Adenoviral Genes

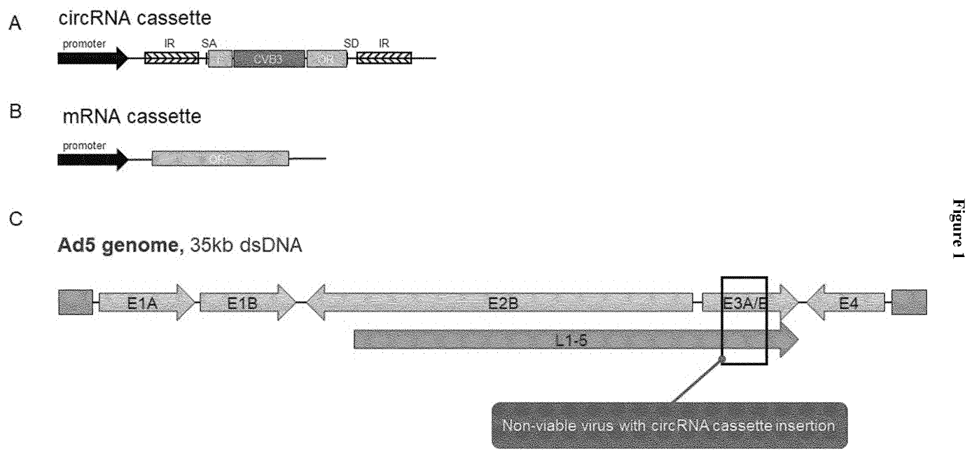

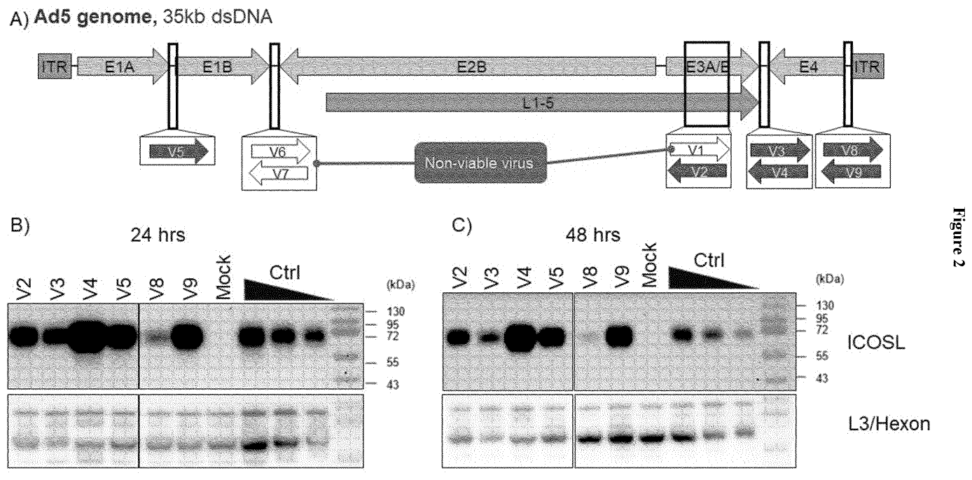

In a first set of experiments a circRNA and mRNA expression cassette (FIG. 1A-B) were inserted into the E3 region of an adenovirus serotype 5 backbone (FIG. 1C). The circRNA cassette is composed of an exogenous CMV promoter, inverted repeat elements, a split ORF, an IRES element and a polyadenylation signal (sequence as in SEQ ID NO:001). The mRNA expression cassette is composed of a CMV promoter, the ORF and a polyadenylation signal.

The adenoviral vector used contains a 2.7 kb deletion within the E3 region to allow for sufficient space for insertion of the payload cassettes within the adenovirus 5 genome. The expression cassette was inserted in the same orientation as the E3 is encoded in the adenoviral genome. Table 2 below indicates the elements encoded in the adenoviral genome.

The inventors observed that insertion of a circular RNA expression cassette within the E3 region inhibited adenovirus vector rescue, while insertion of a linear mRNA expression cassette did not impede viral rescue or fitness. This suggests that insertion of a circular RNA expression cassette has a dramatic effect on viral fitness. Without wishing to be bound by theory the inventors believe that the circRNA expression cassette may contain bona fide splice acceptor and splice donor sequences which may interfere with adenoviral gene splicing. The insertion in the E3 region might therefore potentially interfere with splicing of the L5 fiber gene.

TABLE 2
Elements encoded in Ad5 genome used in the examples

| | TRANSCRIPTION UNIT | | |
|---|---|---|---|
| | (E: EARLY GENES; | | |
| | I: INTERMEDIATE GENES; | | |
| PROTEIN NAME | L: LATE GENES) | | STRAND |
| CONTROL PROTEIN E1A | E1A | (E) | F |
| CONTROL PROTEIN E1B 19K | E1B | (E) | F |
| CONTROL PROTEIN E1B 55K | E1B | (E) | F |
| CAPSID PROTEIN IX | pIX | (I) | F |
| ENCAPSIDATION PROTEIN IVA2 | pIVa2 | (I) | R |
| DNA POLYMERASE | E2B | (E) | R |
| TERMINAL PROTEIN PRECURSOR PTP | E2B | (E) | R |
| ENCAPSIDATION PROTEIN 52K | L1 | (L) | F |
| CAPSID PROTEIN PRECURSOR PIIIA | L1 | (L) | F |
| PENTON BASE (CAPSID PROTEIN III) | L2 | (L) | F |
| CORE PROTEIN PRECURSOR PVII | L2 | (L) | F |
| CORE PROTEIN V | L2 | (L) | F |
| CORE PROTEIN PRECURSOR PX | L2 | (L) | F |
| CAPSID PROTEIN PRECURSOR PVI | L3 | (L) | F |
| HEXON (CAPSID PROTEIN II) | L3 | (L) | F |
| PROTEASE | L3 | (L) | F |
| DNA BINDING PROTEIN | E2A | (E) | R |
| HEXON ASSEMBLY PROTEIN 100K | L4 | (L) | F |
| PROTEIN 33K | L4 | (L) | F |
| ENCAPSIDATION PROTEIN 22K | L4 | (L) | F |
| CAPSID PROTEIN PRECURSOR PVIII | L4 | (L) | F |
| CONTROL PROTEIN E3 12.5K | E3 | (E) | F |
| FIBER (CAPSID PROTEIN IV) | L5 | (L) | F |
| CONTROL PROTEIN E4ORF6/7 | E4 | (E) | R |
| CONTROL PROTEIN E4 34K | E4 | (E) | R |
| CONTROL PROTEIN E4ORF4 | E4 | (E) | R |
| CONTROL PROTEIN E4ORF3 | E4 | (E) | R |
| CONTROL PROTEIN E4ORF2 | E4 | (E) | R |
| CONTROL PROTEIN E4ORF1 | E4 | (E) | R |

### Example 2: Insertion Outside of Transcriptional Units of the Adenovirus Genome

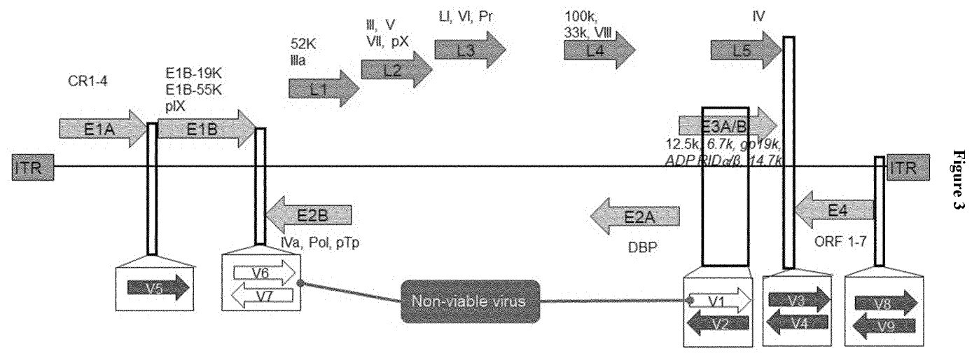

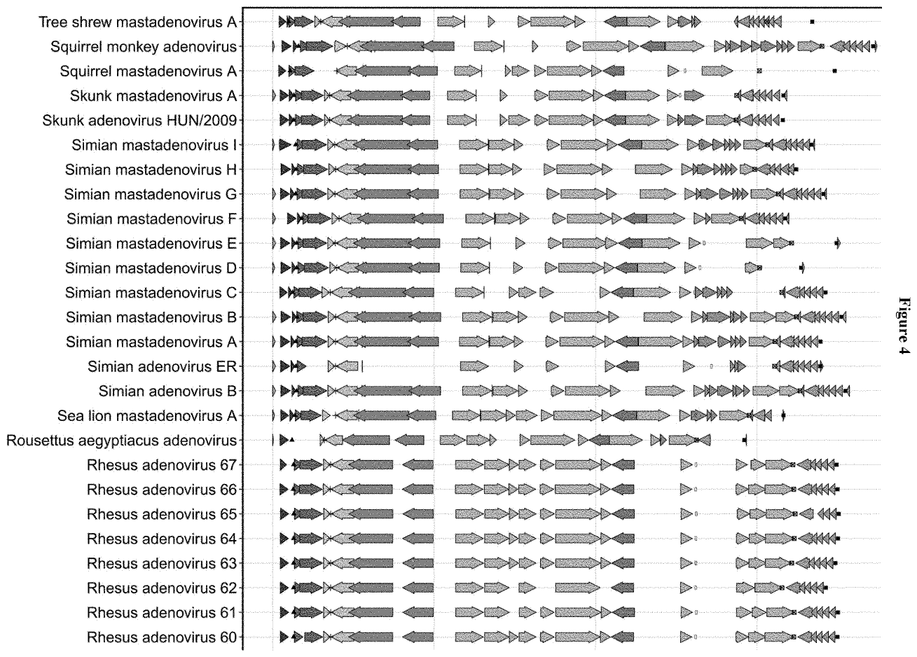

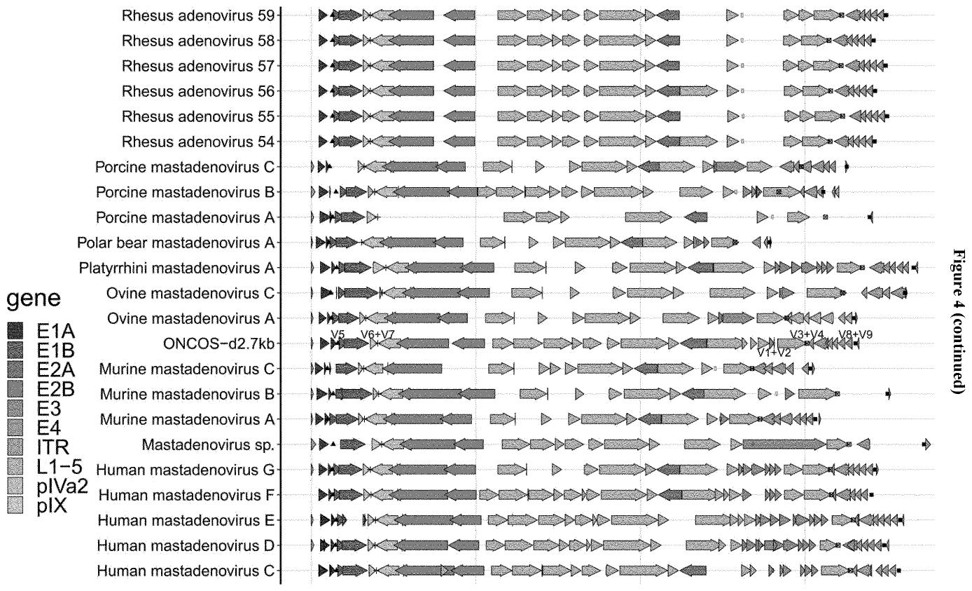

In order to identify suitable integration sites within the adenoviral genome, the inventors inserted circRNA expression cassettes into different regions in the adenovirus 5 genome. Based on the hypothesis that insertion of the circRNA cassette may interfere with splicing of adenoviral genes, the expression cassettes were inserted between transcriptional units within the crAdV5 vector. The circRNA expression cassettes were incorporated in both the forward and reverse orientation at most integration sites (FIG. 2A and Table 3).

TABLE 3
Insertion sites of circRNA cassette within adenovirus 5 backbone

| Insertion | Transcriptional units/genes | | Viable |
|---|---|---|---|
| site | flanking insertion site | Orientation | Virus |
| V1 | E3 | Forward | No |
| V2 | E3 | Reverse | Yes |
| V3 | L5/E4 | Forward | Yes |
| V4 | L5/E4 | Reverse | Yes |
| V5 | E1A/E1B | Forward | Yes |
| V6 | pIX/IVa2 | Forward | No |
| V7 | pIX/IVa2 | Reverse | No |
| V8 | E4/ITR | Forward | Yes |
| V9 | E4/TTR | Reverse | Yes |

The insertion point used to insert the expression cassette were selected to ideally avoid overlap with a transcriptional unit on both strands, but at least avoid overlap with transcriptional units encoded on the same strand. The distances of the insertion point to the closest transcriptional units on either strand is depicted in Table 4 below.

TABLE 4
Relative location of insertion sites

| | | Upstream (5′) | | Downstream (3′) | |
|---|---|---|---|---|---|
| Insertion | | distance to closest | | distance to closest | |
| site | Strand | transcriptional unit | | transcriptional unit | |
| V1^{#} | F | 595 bp | (E3 TATA) | 31 bp | (E3 PAS) |
| V2 | R | 2025 bp | (E4 PAS) | 1057 bp | (E2a promoter) |
| V3 | F | 10 bp | (L5 PAS) | 24 bp | (*E4 PAS) |
| V4 | R | 24 bp | (E4 PAS) | 10 bp | (*L5 PAS) |
| V5 | F | 25 bp | (E1A PAS) | 30 bp | (E1B TATA) |
| V6 | F | 23 bp | (pIX PAS) | 23 bp | (*pIVa PAS) |
| V7 | R | 23 bp | (pIV PAS) | 23 bp | (*pIX PAS) |
| V8 | F | 97 bp | (*E4 TATA) | 97 bp | (ITR) |
| V9 | R | 97 bp | (ITR) | 97 bp | (E4 TATA) |

*denotes opposite reading direction as inserted expression cassette (i.e. encoded on the opposite strand of the adenovirus genome;
^{#}V1 is inserted within a transcriptional unit and the above distances thus indicate the beginning and end of the transcriptional unit in which V1 is inserted.

These distances were calculated based on the start and end of the closest transcriptional unit, which were defined as starting with a TATA box and ending with a polyadenylation signal (PAS). The relevant element used to calculate the distance in Table 4 is indicated next to the distance in base pairs (bp).

The choice of insertion site had a dramatic effect on virus viability (Table 3). For insertion sites V6 and V7 the expression cassette was placed between the pIX and IVa ORF, but no viable virus was able to be generated (FIG. 2B-C).

In addition to the general suitability of the insertion site to recover viable virus particles the inventors further unexpectedly found that the sites differed significantly with regard to expression strength of the circRNA construct. CircRNA-derived protein expression was assessed by western blot 24 h and 48 h post-infection of A549 cells with an equal number of viral particles.

CircRNA-derived protein expression differed between each insertion site, with the highest yield arising from insertion of the circRNA cassette between the L5 and E4 region in the reverse orientation (V4) (FIG. 2B-C). Comparable payload expression was observed between viruses V2, V5 and V9 (FIG. 2B-C). Low levels of protein expression were observed for virus V3 and V8 (FIG. 2B-C).

The inventors further observed that expression cassettes inserted in an orientation in which the direction of transcription (i.e. 5′ to 3′) is not towards the closest ITR of the

### Example 3: Insertion Sites are Conserved in Mastadenovirus Family

The inventors further investigated if the identified insertion sites are specific for Adenovirus Ad5 or if these findings can be extrapolated to at least the Mastadenovirus family. Therefore an alignment of accessible Mastadenovirus genomes was performed to test whether these sites are also present in the other Mastadenovirus genomes.

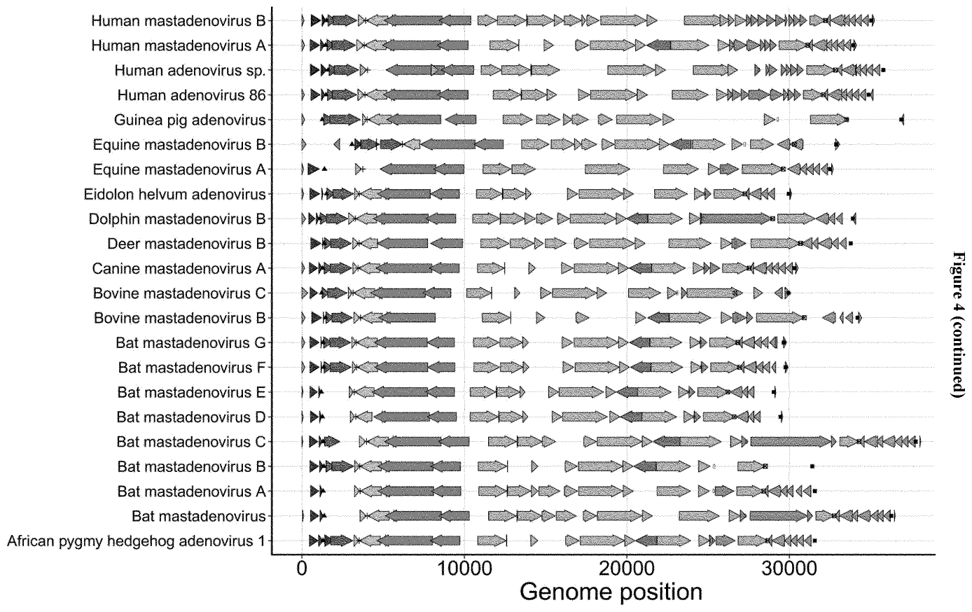

The alignment including the annotation of relevant transcriptional units and genes is depicted in FIG. 4. The adenovirus 5 genome used in the previous experiments is labelled ‘ONCOS-d2.7 kb’ and the different insertion sites are depicted in this genome. As can be easily seen the transcriptional units present in Ad5 are also present in the other Mastadenovirus with similar positions relative to each other. Thus the same sites between transcriptional units tested and verified in Ad5 are present and conserved in the other members of the Mastadenovirus family. The insertion strategy used for adenovirus 5 (Ad5) in the above examples can thus be transferred to the other Mastadenovirus family members using the conserved genome structure.

### SUMMARY

The disclosed experimental data demonstrates that expression of a circular RNA encoding cassette from an adenoviral genome is complex. Unlike linear mRNA expression cassettes insertion of the circRNA expression cassettes into coding regions of the genome greatly impacts viral fitness, for example when inserted into the E3 region.

Additionally, the inventors observed that placing the circRNA expression cassette between (and thereby outside of) these coding regions and transcriptional units is not sufficient to ensure circRNA expression. As observed for insertion sites V6 and V7 insertion of the circRNA between the pIX and IVa genes did not give rise to viable virus.

In summary the present invention identified specific regions in the adenovirus genome which are permissible to insertion of the circRNA expression cassette. Furthermore, the inventors also identified specific regions that allow for high yield circRNA expression.

1. An adenovirus genome encoding an adenovirus, comprising at least one heterologous expression cassette comprising or consisting of a nucleic acid sequence encoding a circular RNA (circRNA), wherein the heterologous expression cassette is inserted outside of functional transcriptional units of the adenovirus genome having the same reading direction as the inserted heterologous expression cassette.

2. The adenovirus genome of claim 1, wherein the adenovirus is a conditionally replicating adenovirus.

3. The adenovirus genome according to claim 1, wherein the heterologous expression cassette is inserted at a point in the adenovirus genome having a distance of at least 24 bp to a transcriptional unit encoding an intermediate gene.

4. The adenovirus genome according to claim 1, wherein the distance of the heterologous expression cassette to the inverted terminal repeat (ITR) of the adenovirus genome is shorter upstream than downstream of the heterologous expression cassette.

5. The adenovirus genome according to claim 1, wherein the inverted terminal repeat (ITR) of the adenovirus genome is within 6000 nucleotides upstream of the heterologous expression cassette.

6. The adenovirus genome according to claim 1, wherein the heterologous expression cassette is inserted between transcriptional units E1A/E1B, E4/E2A, L5/E4 or between E4 and the 3′-terminal ITR.

7. The adenovirus genome according to claim 1, wherein the adenovirus genome is a Mastadenovirus genome, preferably a human or great ape adenovirus genome, more preferably a group C adenovirus genome.

8. The adenovirus genome according to claim 7, wherein the adenovirus genome is a genome of human adenovirus 5 (hAd5).

9. The adenovirus genome according to claim 1, wherein the heterologous expression cassette comprises an open reading frame (ORF) encoding a circular RNA of interest and an internal ribosome entry site (IRES) operably linked to the ORF.

10. The adenovirus genome according to claim 1, wherein the circular RNA of interest in the heterologous expression cassette is a transgene.

11. The adenovirus genome according to claim 1, wherein the heterologous expression cassette further comprises:
a first and a second backsplicing site allowing for circularization of the circRNA;
a promoter operably linked to the ORF;
a first and a second inverted repeat (IR) element; and/or
a polyadenylation signal.

12. An infectious, preferably conditionally replicative, adenovirus particle comprising the adenovirus genome of claim 1.

13. A pharmaceutical composition comprising the adenovirus genome of claim 1 and a pharmaceutically acceptable carrier and/or excipient.

14. (canceled)

15. A method for therapy or prophylaxis of proliferative diseases, comprising administering to a subject in need thereof the adenovirus genome of claim 1.


SEQUENCE LISTING (18 sequences)

SEQ ID NO: 1 - DNA, 3535 bp, synthetic construct
  source 1..3535: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcgtactttttttttgagacagagtcttgctctgtctcacaggctgaagtgcagtggtgcaatctcggctcactgcaacctccacctcccaggttcaagcgattctcctccctcagcctcccgagtagctgggaccacaggcatgcaccaccatccccagctaatttttgcattattagtagagttgggatttcttcaccgtgttggccaggctggtcttggactcctgacctcaagtgatccaactgcctcagcctctcaaagtgctaggattacagggcatgagccaaagcacccggcatctatacttttcttttgagggaaaatgttggcaccgtttctttcctgatatttaaaattgaaaaaaaaattgttgacattaatatttcttctttccttttttttcttttcctttttttttttttttttgcaggacgagcagaagtttcactgcctggtgttgagccaatccctgggattccaggaggttttgagcgttgaggttacactgcatgtggcagcaaacttcagcgtgcccgtcgtcagcgccccccacagcccctcccaggatgagctcaccttcacgtgtacatccataaacggctaccccaggcccaacgtgtactggatcaataagacggacaacagcctgctggaccaggctctgcagaatgacaccgtcttcttgaacatgcggggcttgtatgacgtggtcagcgtgctgaggatcgcacggacccccagcgtgaacattggctgctgcatagagaacgtgcttctgcagcagaacctgactgtcggcagccagacaggaaatgacatcggagagagagacaagatcacagagaatccagtcagtaccggcgagaaaaacgcggccacgtggagcatcctggctgtcctgtgcctgcttgtggtcgtggcggtggccataggctgggtgtgcagggaccgatgcctccaacacagctatgcaggtgcctgggctgtgagtccggagacagagctcactggccacgtttgaatgcggctgggcagtcctggactgctcttcctgctcttcagcagccttcgagctgatactcaggagaaggaagtcagagcgatggtaggcagcgacgtggagctcagctgcgcttgccctgaaggaagccgttttgacttaaatgatgtttacgtatattggcaaaccagtgagtcgaaaaccgtggtgacctaccacatcccacagaacagctccttggaaaacgtggacagccgctaccggaaccgagccctgatgtcaccggccggcatgctgcggggcgacttctccctgcgcttgttcaacgtcaccccccagttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatgcggctgggcagtcctggactgctcttcctgctcttcagcagccttcgagctgatactcaggagaaggaagtcagagcgatggtaggcagcgacgtggagctcagctgcgcttgccctgaaggaagccgttttgacttaaatgatgtttacgtatattggcaaaccagtgagtcgaaaaccgtggtgacctaccacatcccacagaacagctccttggaaaacgtggacagccgctaccggaaccgagccctgatgtcaccggccggcatgctgcggggcgacttctccctgcgcttgttcaacgtcaccccccaggtaggtaacaactccatactttttggttgtttattaatgtgaaatttctgctaaatgaaatacttttgtgtgtgtttgtggtagaagagaccacttcagttaaataaggaaatcaagagaggatcaatttaggttcgttttaccgggtgcagtggctcatgcctgtaatccctgcacttagggaggctgaggcgggtggatgacctgaggttaggagttcaagaccagcctggccaacatggcgaaacccccatctctactaaaaataacaaaaattagctgggtgtggtggtgggtgtctataatcccagcaacttgggaggctgaggcaggagaatcacttgaacccaggagatggaggttgcagtgagccgagatcatgccattgcactccagcaacttgtttattgcagcttataatggttacaaataaagcaatagcatcacaaatttcacaaataaagcatttttttcactgcattctagttgtggtttgtccaaactcatcaatgtatctta

SEQ ID NO: 2 - DNA, 37 bp, synthetic construct
  source 1..37: mol_type = other DNA
  regulatory 20..23: regulatory_class = TATA_box
agctgacgtgtagtgtatttatacccggtgagttcct

SEQ ID NO: 3 - DNA, 32 bp, synthetic construct
  source 1..32: mol_type = other DNA
  regulatory 13..16: regulatory_class = TATA_box
gggcttaaagggtatataatgcgccgtgggct

SEQ ID NO: 4 - DNA, 35 bp, synthetic construct
  source 1..35: mol_type = other DNA
  regulatory 14..17: regulatory_class = TATA_box
gtgggaaagaatatataaggtgggggtcttatgta

SEQ ID NO: 5 - DNA, 34 bp, synthetic construct
  source 1..34: mol_type = other DNA
  regulatory 15..18: regulatory_class = CAAT_signal
ctacacctacaaaccaatcaccttccttgatgcc

SEQ ID NO: 6 - DNA, 29 bp, synthetic construct
  source 1..29: mol_type = other DNA
  regulatory 11..14: regulatory_class = TATA_box
gaaggggggctataaaagggggtgggggc

SEQ ID NO: 7 - DNA, 28 bp, synthetic construct
  source 1..28: mol_type = other DNA
  regulatory 14..17: regulatory_class = TATA_box
cgcccgggcagggtataactcacctgac

SEQ ID NO: 8 - DNA, 29 bp, synthetic construct
  source 1..29: mol_type = other DNA
  regulatory 14..18: regulatory_class = CAAT_signal
cgcgaaactagtccttaagagtcagcgcg

SEQ ID NO: 9 - DNA, 31 bp, synthetic construct
  source 1..31: mol_type = other DNA
  regulatory 15..18: regulatory_class = TATA_box
tcattttttagtcctatatatactcgctctg

SEQ ID NO: 10 - DNA, 37 bp, synthetic construct
  source 1..37: mol_type = other DNA
  regulatory 18..23: regulatory_class = polyA_signal_sequence
tgagttgatgtaagtttaataaagggtgagataatgt

SEQ ID NO: 11 - DNA, 37 bp, synthetic construct
  source 1..37: mol_type = other DNA
  regulatory 17..22: regulatory_class = polyA_signal_sequence
tgcggtttaaaacataaataaaaaaccagactctgtt

SEQ ID NO: 12 - DNA, 37 bp, synthetic construct; identical to SEQ ID NO: 11
  source 1..37: mol_type = other DNA
  regulatory 17..22: regulatory_class = polyA_signal_sequence

SEQ ID NO: 13 - DNA, 31 bp, synthetic construct
  source 1..31: mol_type = other DNA
  regulatory 13..18: regulatory_class = polyA_signal_sequence
gcaaaacccctaaataaagacagcaagacac

SEQ ID NO: 14 - DNA, 28 bp, synthetic construct
  source 1..28: mol_type = other DNA
  regulatory 13..18: regulatory_class = polyA_signal_sequence
gagaagatgactaataaagaatcgtttg

SEQ ID NO: 15 - DNA, 28 bp, synthetic construct
  source 1..28: mol_type = other DNA
  regulatory 12..17: regulatory_class = polyA_signal_sequence
taaaaaaaaataataaagcatcacttac

SEQ ID NO: 16 - DNA, 31 bp, synthetic construct; identical to SEQ ID NO: 13
  source 1..31: mol_type = other DNA
  regulatory 13..18: regulatory_class = polyA_signal_sequence

SEQ ID NO: 17 - DNA, 30 bp, synthetic construct
  source 1..30: mol_type = other DNA
  regulatory 12..17: regulatory_class = polyA_signal_sequence
tgcaattgaaaaataaacacgttgaaacat

SEQ ID NO: 18 - DNA, 12 bp, synthetic construct
  source 1..12: mol_type = other DNA
natgccaatgcn